(19) United States
(12) Patent Application Publication
(10) Pub. No.: US 2017/0143385 A1
(43) Pub. Date: May 25, 2017

(54) SPINAL ROD REDUCTION SYSTEM

(71) Applicant: K2M, Inc., Leesburg, VA (US)

(72) Inventors: Ashok Biyani, Toleda, OH (US); Geneva Goldwood, Leesburg, VA (US); Josh Rubin, McLean, VA (US)

(21) Appl. No.: 15/357,053

(22) Filed: Nov. 21, 2016

Related U.S. Application Data
(60) Provisional application No. 62/257,403, filed on Nov. 19, 2015.

(51) Int. Cl. A61B 17/70

(52) U.S. Cl. CPC A61B 17/7086; A61B 17/7032

(57) ABSTRACT

A spinal rod reduction apparatus including an elongated body member, a pair of arms extending distally from the elongated body member, and first and second elongated slots defined between the pair of arms. The first and second elongated slots are configured to receive a spinal rod therethrough. The first elongated slot is positioned in opposed relation to the second elongated slot and is longer than the second elongated slot.

### CROSS REFERENCE TO RELATED APPLICATIONS

This application claims the benefit of U.S. Provisional Patent Application Ser. No. 62/257,403, filed Nov. 19, 2015, the entire contents of which are incorporated by reference herein.

### TECHNICAL FIELD

The present disclosure relates to spinal surgery. More specifically, the present disclosure relates to systems, devices, and methods for reducing spinal rods into pedicle screw housings.

### BACKGROUND

The spinal column is a complex system of bones and connective tissues that provide support for the human body and protection for the spinal cord and nerves. The adult spine is comprised of an upper and lower portion. The upper portion contains 24 discrete bones, which are subdivided into three areas including 7 cervical vertebrae, 12 thoracic vertebrae and 5 lumbar vertebrae. The lower portion is comprised of the sacral and coccygeal bones. The cylindrical shaped bones, called vertebral bodies, progressively increase in size from the upper portion downwards to the lower portion.

An intervertebral disc along with two posterior facet joints cushion and dampen the various translational and rotational forces exerted upon the spinal column. The intervertebral disc is a spacer located between two vertebral bodies. The facets provide stability to the posterior portion of adjacent vertebrae. The spinal cord is housed in the canal of the vertebral bodies. It is protected posteriorly by the lamina. The lamina is a curved surface with three main protrusions. Two transverse processes extend laterally from the lamina, while the spinous process extends caudally and posteriorly. The vertebral bodies and lamina are connected by a bone bridge called the pedicle.

The spine is a flexible structure capable of a large range of motion. There are various disorders, diseases, and types of injury which restrict the range of motion of the spine or interfere with important elements of the nervous system. The problems include, but are not limited to scoliosis, kyphosis, excessive lordosis, spondylolisthesis, slipped or ruptured discs, degenerative disc disease, vertebral body fracture, and tumors. Persons suffering from any of the above conditions may experience extreme or debilitating pain and diminished nerve function. These conditions and their treatments can be further complicated if the patient is suffering from osteoporosis, or bone tissue thinning and loss of bone density.

Spinal fixation apparatuses are widely employed in surgical procedures for correcting spinal injuries and diseases. When the disc has degenerated to the point of requiring removal, there are a variety of interbody implants that are utilized to take the place of the disc. These include polyetheretherketone (“PEEK”) interbody spacers, metal cages, and cadaver and human bone implants. In order to facilitate stabilizing the spine and keeping the interbody in position, other implants are commonly employed, including longitudinally linked rods secured to coupling elements, which in turn are secured to the bone by spinal bone fixation fasteners such as pedicle screws, hooks, and others. The opposing pair of longitudinally linked rods is commonly disposed along the long axis of the spine via a posterior approach. Pedicle screws are utilized to capture these rods and can be manufactured from any biocompatible material, including cobalt chrome, stainless steel and titanium. It is desired to perform these procedures in a minimally invasive manner to minimize pain and reduce recovery time for the patient.

### SUMMARY

Accordingly, one aspect of the present disclosure is directed to a spinal rod reduction system. The spinal rod reduction system includes a bone anchor and a reduction tower. The bone anchor includes a housing and a threaded shank extending from the housing. The housing defines an opening configured to receive a spinal rod therein. The bone anchor may be configured to receive a set screw to selectively secure a spinal rod within the opening of the housing.

The reduction tower has a pair of arms releasably attachable to the housing of the bone anchor. The pair of arms defines first and second elongated slots between the pair of arms. The first and second elongated slots are configured to receive a spinal rod therethrough and direct a spinal rod toward the opening of the housing of the bone anchor. The first elongated slot is positioned in opposed relation to the second elongated slot. The first elongated slot may be longer than the second elongated slot. The first and second elongated slots may be aligned with the opening of the housing while the reduction tower is attached to the bone anchor.

In certain embodiments, a rod reducer may selectively couple to the reduction tower to reduce a spinal rod along the first and second elongated slots and into the opening of the housing.

In some embodiments, the reduction tower includes a threaded proximal end configured to threadably engage the rod reducer.

The spinal rod reduction system may further include a spinal rod.

In certain embodiments, the spinal rod reduction system includes a tower removal instrument engagable with the reduction tower and configured to separate the reduction tower from the bone anchor.

In accordance with another embodiment of the present disclosure, a spinal rod reduction apparatus includes an elongated body member, a pair of arms extending distally from the elongated body member, and first and second elongated slots defined between the pair of arms. The first and second elongated slots are configured to receive a spinal rod therethrough. The first elongated slot is positioned in opposed relation to the second elongated slot. The first elongated slot is longer than the second elongated slot.

In some embodiments, the pair of arms may be configured to couple to a bone anchor defining an opening for receiving a spinal rod therein. The first and second elongated slots may be positioned to align with the opening of the bone anchor while the pair of arms is coupled to the bone anchor.

In embodiments, the pair of arms extends to a distal end that is engagable with an outer surface of the bone anchor.

In some embodiments, the pair of arms are positioned to receive a set screw between the pair of arms. The set screw may be advanceable along an inner surface of the pair of arms to reduce a spinal rod received between the pair of arms.

In certain embodiments, a proximal end of the elongated body member is selectively engagable with a rod reducer. The proximal end of the elongated body member may be threaded.

In some embodiments, the pair of arms and the body member may be integrally formed.

According to another aspect of the present disclosure, a method of reducing a spinal rod is provided. The method includes securing a bone anchor and a reduction tower to a vertebra, positioning the spinal rod through a first elongated slot of the reduction tower, advancing the spinal rod along the first elongated slot toward a second elongated slot, advancing the spinal rod through the second elongated slot, and advancing the spinal rod along the first and second elongated slots and towards an opening of the bone anchor.

The method may involve securing the reduction tower to the bone anchor before the bone anchor is secured to the vertebra.

The method may further include securing a rod reducer to the reduction tower.

The method may further include advancing the spinal rod along the reduction tower by rotating a proximal end of the rod reducer around a proximal end of the reduction tower.

The method may involve separating the reduction tower from the bone anchor after the spinal rod is reduced into the bone anchor. Separating the reduction tower from the bone anchor may include coupling a tower removal instrument to the reduction tower.

Other aspects, features, and advantages will be apparent from the description, the drawings, and the claims that follow.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description given below, serve to explain the principles of the disclosure, wherein:

FIG. 1A is a front view of one embodiment of a spinal rod reduction assembly;

FIG. 1B is a side view of the spinal rod reduction assembly of FIG. 1A;

FIG. 2 is front view, with parts separated, of the spinal rod reduction assembly of FIGS. 1A and 1B;

FIG. 3 is cross-sectional view of the spinal rod reduction assembly of FIGS. 1A and 1B as taken along section line 3-3 shown in FIG. 1A;

FIG. 4 is a cross-sectional view of the spinal rod reduction assembly of FIGS. 1A and 1B as taken along section line 4-4 shown in FIG. 1B;

FIGS. 5 and 6 are progressive, perspective views illustrating distal advancement of a spinal rod along the spinal rod reduction assembly of FIGS. 1A and 1B;

FIG. 7 is a perspective view of one embodiment of a rod reducer;

FIG. 8 is a side view of the rod reducer of FIG. 7;

FIGS. 9 and 10 are progressive, side views illustrating the rod reducer of FIGS. 7 and 8 reducing a spinal rod into an opening of a bone anchor of the spinal rod reduction assembly of FIGS. 1A and 1B;

FIG. 11 is a perspective view of another embodiment of a rod reducer;

FIG. 12 is a cross-sectional view of the rod reducer of FIG. 11 as taken along section line 12-12 shown in FIG. 11;

FIGS. 13 and 14 are progressive, perspective views illustrating the rod reducer of FIG. 11 reducing a spinal rod into the opening of the bone anchor of the spinal rod reduction assembly of FIGS. 1A and 1B;

FIG. 15 is a side view of a tower removal instrument shown in a first position;

FIG. 16 is a side view of the tower removal instrument of FIG. 15 shown in a second position; and

FIG. 17 is a longitudinal, cross-sectional view of the tower removal instrument as taken through a plane extending along a central longitudinal axis of the tower removal instrument, the tower removal instrument shown in the second position.

### DETAILED DESCRIPTION

Embodiments of the presently disclosed devices are described in detail with reference to the drawings, in which like reference numerals designate identical or corresponding elements in each of the several views. As used herein, the term “distal” or “leading” refers to that portion of the device that is farther from the user, while the term “proximal” or “trailing” refers to that portion of the device that is closer to the user. As used herein, the term “clinician” refers to a doctor, nurse, or other care provider and may include support personnel. In the following description, well-known functions or construction are not described in detail to avoid obscuring the present disclosure in unnecessary detail.

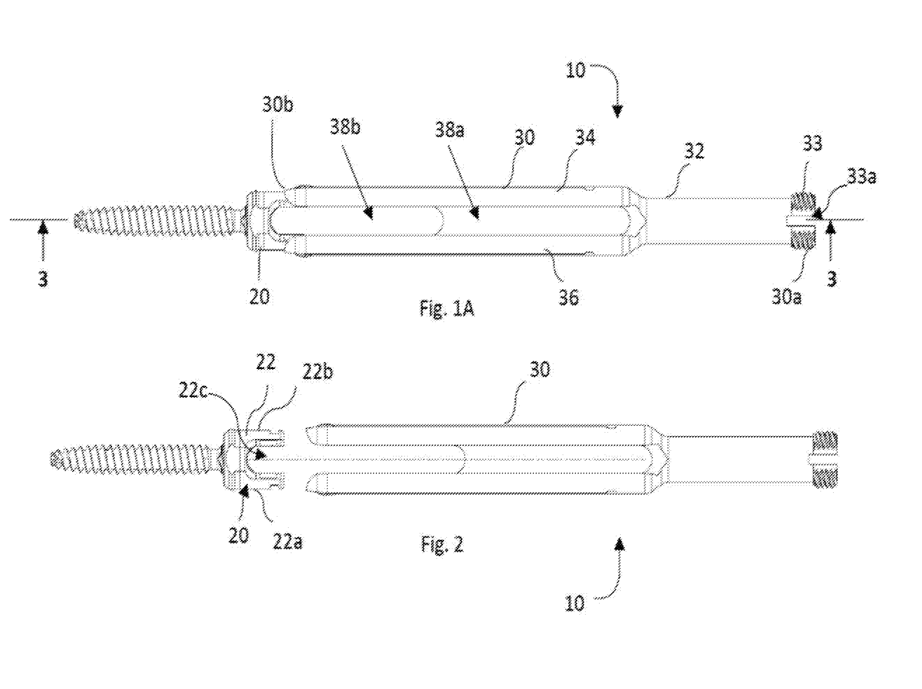

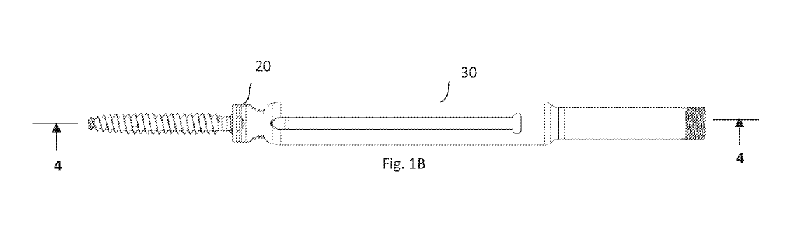

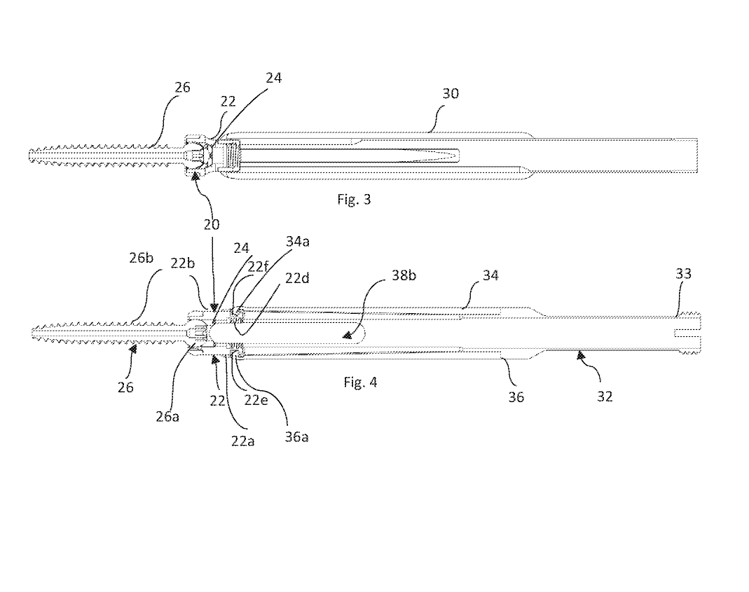

With reference to FIGS. 1A-6, a spinal rod reduction assembly 10 includes a pedicle screw or bone anchor 20 and a reduction tower 30.

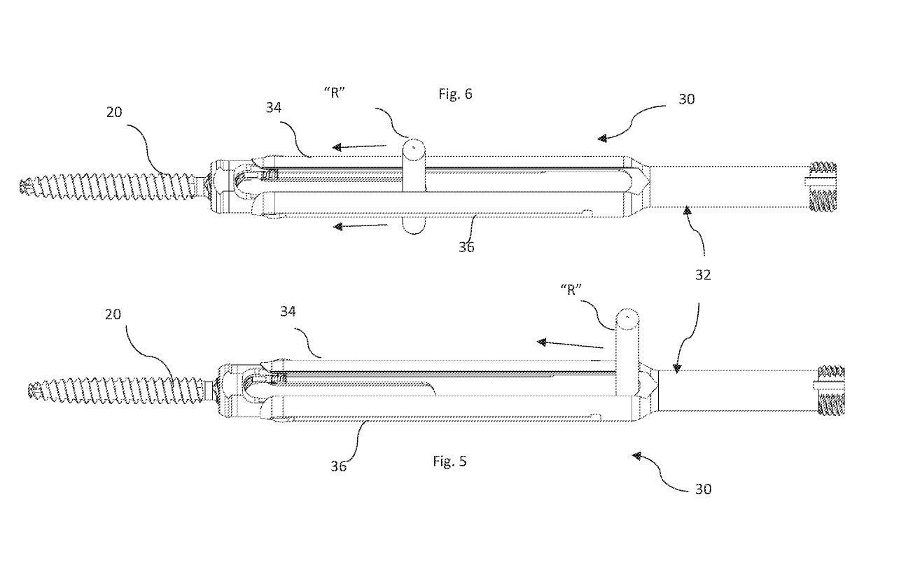

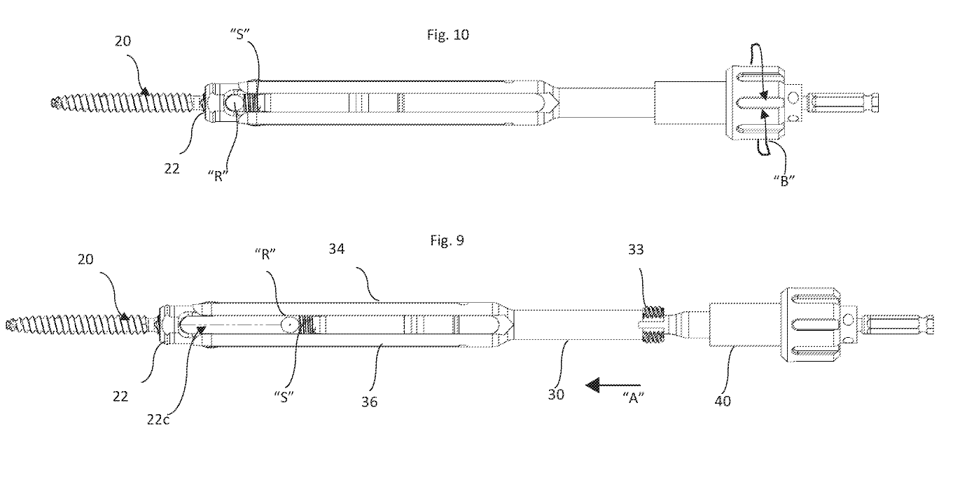

In general, the bone anchor 20 includes a housing 22, an anvil 24 received in the housing 22 to support a spinal rod “R” (FIGS. 5-6) in the housing 22, and a bone screw 26 extending from the housing 22. The housing 22 includes opposing wings or walls 22a, 22b that define an opening or U-shaped channel 22c between the walls 22a, 22b that receives the spinal rod “R.” The walls 22a, 22b include a threaded inner surface 22d that receives a set screw “S” (FIG. 9) to secure the spinal rod “R” within the housing 22. The walls 22a, 22b include flanged recess 22e, 22f, respectively, that facilitate grasping of the housing 22 by the reduction tower 30 or other instrument. The bone screw 26 includes a head 26a supported in the housing 22 beneath the anvil 24 and a threaded shank 26b that extends distally from the housing 22.

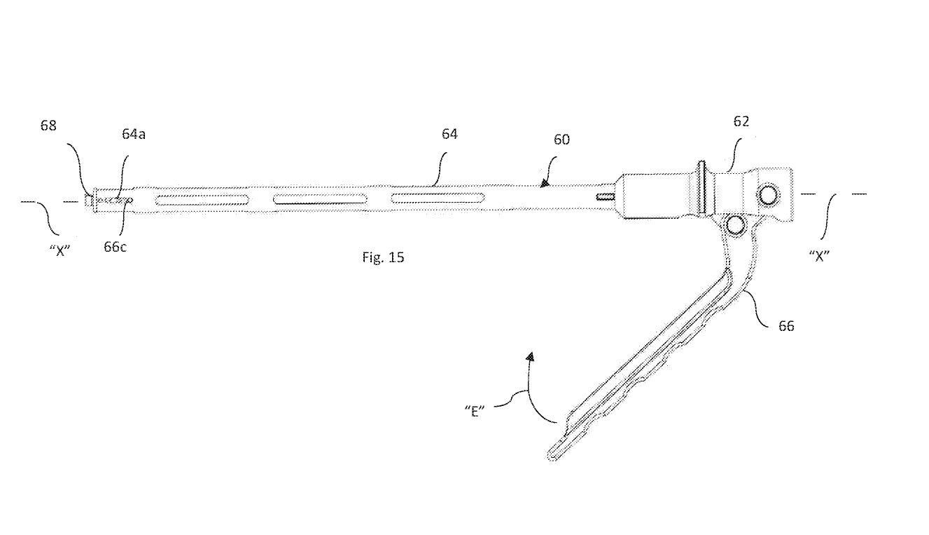

The reduction tower 30 has a proximal end 30a and a distal end 30b and includes body 32. The proximal end 30a of the reduction tower 30 includes a threaded portion 33 separated by one or more slots 33a. The slots 33a may be disposed on opposed sides of the proximal end 30a of the reduction tower 30 in mirrored relation. The slots 33a enable a tower removal instrument 60 (see FIGS. 15-17) to align with, and couple to, the reduction tower 30 for separating the reduction tower 30 from the bone anchor 20 as described in greater detail below. First and second arms 34, 36 extend distally from a distal end of the body 32 to the distal end 30b of the reduction tower 30. The first and second arms 34, 36 have engagement ends 34a, 36a at respective distal ends of the first and second arms 34, 36. The first and second arms 34, 36 define first and opposed elongated slots 38a, 38b that extend to the distal end 30b of the reduction tower 30. The second elongated slot 38b is shorter than the first elongated slot 38a to facilitate spinal rod “R” passage. In general, the first and second elongated slots 38a, 38b are arranged to guide the spinal rod “R” to facilitate insertion/adjustment of the spinal rod “R” with respect to the bone anchor 20 so as to achieve desired positioning of the spinal rod “R,” the bone anchor 20 (or multiple bone anchors 20), and/or a spine/vertebral body (not shown). The first elongated slot 38a is taller (e.g., higher) than the second elongated slot 38b to make it easier for a clinician to locate the first elongated slot 38a when starting spinal rod “R” passage above a subject's skin. The second elongated slot 38b is shorter (e.g., lower) than the first elongated slot 38a to help guide the spinal rod “R” below the fascia and musculature of the subject so that it is easier (e.g., requires less reduction) to seat the spinal rod “R” into the U-shaped channel 22c of the housing 22 of the bone anchor 20.

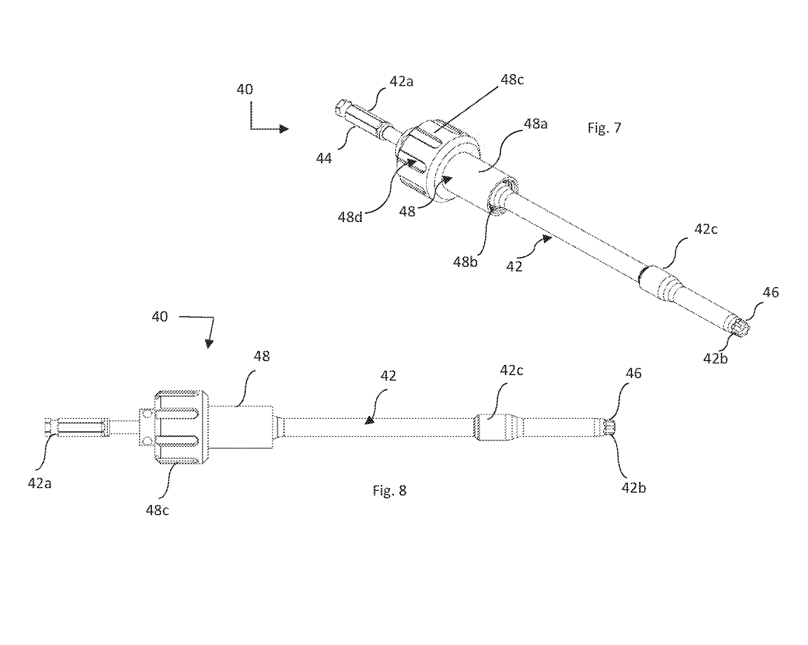

Turning now to FIGS. 7-10, one embodiment of a rod reduction device 40 for use with spinal rod reduction assembly 10 is provided. The rod reduction device 40 includes an elongated shaft 42 that extends from a proximal end 42a to a distal end 42b. The proximal end 42a of the elongated shaft 42 supports an attachment member 44 and the distal end 42b of the elongated shaft 42 includes a driving tip 46 configured to engage a set screw “S.” The driving tip 46 may include a hexolobular configuration. The elongated shaft 42 further includes a bulbous portion 42c projecting radially outwardly from the elongated shaft 42 and configured to assert a separation force against inner surfaces of the first and second arms 34, 36 of the reduction tower 30 to maintain the first and second arms 34, 36 separated a predetermined distance while the rod reduction device 40 advances the set screw “S” through the rod reduction tower 30 as shown in FIGS. 9 and 10. A sleeve 48 is mounted over the elongated shaft 42 and includes an outer surface 48a and a threaded inner surface 48b. The outer surface 48a of the sleeve 48 includes a manual drive collar 48c including gripping grooves 48d defined therein to facilitate gripping.

In use, as seen in FIGS. 9 and 10, with the bone anchor 20 secured to bone (not shown) with the reduction tower 30 mounted thereto, the rod reduction device 40 can be inserted into the proximal end 30a of the reduction tower 30, as indicated by arrow “A,” so that the driving tip 46 of the rod reduction device 40 can engage the set screw “S” received between the first and second arms 34, 36 of the reduction tower 30. Once the threaded inner surface 48b (FIG. 7) of the sleeve 48 of the rod reduction device 40 engages the threaded portion 33 of the rod reduction tower 30, the rod reduction device 40 can then be rotated, as indicated by arrows “B,” relative to the rod reduction tower 30 until the spinal rod “R” is reduced into the U-shaped channel 22c of the housing 22 of the bone anchor 20 with the set screw “S” threadably securing the spinal rod “R” therein. Rotation/torque can be provided (e.g., manually) to the elongated shaft 42 and the driving tip 46 through the drive collar 48c and/or the attachment end 44 of the rod reduction device 40. As necessary or desired, an instrument (not shown) such as a handle or the like can be secured to the attachment end 44 of the rod reduction device 40 to provide additional torque to the elongated shaft 42/driving tip 46 thereof for reducing the spinal rod “R” into the U-shaped channel 22c of the bone anchor 20. The rod reduction device 40 and the reduction tower 30 can then be separated from the bone anchor 20, set screw “S,” and spinal rod “R.”

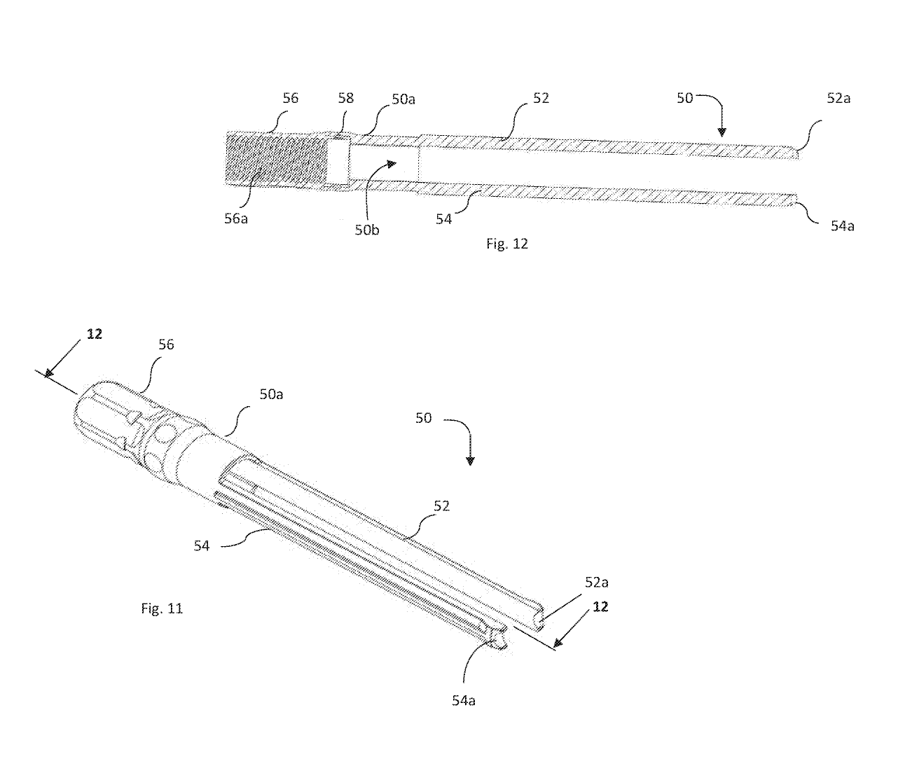

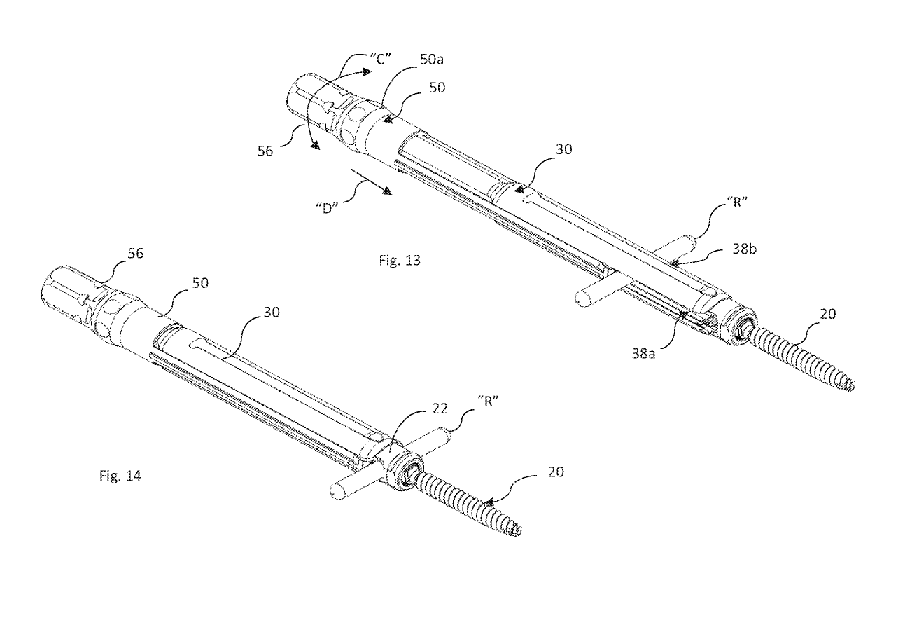

Referring now to FIGS. 11-14, another embodiment of a rod reduction device 50 for use with spinal rod reduction assembly 10 is provided. The rod reduction device 50 includes an elongated tubular body 50a defining a central passage 50b therethrough. The elongated tubular body 50a has a first arm 52 and a second arm 54 extending distally therefrom. The first and second arms 52, 54 are disposed in parallel, spaced apart relation relative to one another. The first and second arms 52, 54 extend distally to rod receiving arches 52a, 54a, respectively, configured to engage a spinal rod “R.” A tubular knob 56 extends proximally from the tubular body 50a and is rotatably connected to the tubular body 50a by a retaining ring 58 that enables the tubular knob 56 to rotate about the tubular body 50a, as indicated by arrows “C.” The tubular knob 56 includes a threaded internal surface 56a.

In use, the rod reduction device 50 is mounted over the reduction tower 30 so that the first and second arms 52, 54 of the rod reduction device 50 are aligned with the first and second elongated slots 38a, 38b of the reduction tower 30, respectively. The rod reduction device 50 is approximated toward the reduction tower 30 until the threaded internal surface 56a of the rod reduction device 50 threadably engages the threaded portion 33 (FIG. 1A) of the reduction tower 30. With a spinal rod “R” inserted through the first and second elongated slots 38a, 38b of the reduction tower 30 and the rod receiving arches 52a, 54a of the rod reduction device 50 engaged with the spinal rod “R,” the tubular knob 56 of the rod reduction device 50 can be rotated relative to the elongated tubular body 50a, as indicated by arrows “C,” to advance the rod reduction device 50 distally relative to the reduction tower 30, as indicated by arrow “D.” As the rod reduction device 50 advances distally relative to the reduction tower 30, the spinal rod “R” reduces into the housing 22 of the bone anchor 20. A set screw “S” (see FIG. 9) can then be secured to the housing 22 of the bone anchor 20 to secure the spinal rod “R” to the bone anchor 20. The set screw “S” may be advanced through the rod reduction device 50 and/or the reduction tower 30 as desired. Alternatively, the rod reduction device 50 and/or the reduction tower 30 may be removed and then the set screw “S” may be secured to the bone anchor 20.

For a detailed discussion of relevant bone anchors and rod reducers, each of which, and/or one or more components thereof, can be utilized in connection with, and/or modified for use with, the presently disclosed devices and systems, reference can be made to U.S. Pat. No. 8,308,729 and U.S. Pat. App. Pub. No. 2013/0046345, the entire contents of each of which are incorporated by reference herein.

Turning now to FIGS. 15-17, a tower removal instrument 60 for use with the spinal rod reduction assembly 10 is provided. The tower removal instrument 60 includes a handle assembly 62, a tubular body 64 extending distally from the handle assembly 62, and an actuation assembly 66 coupled to the handle assembly 62. A distal tip 68 is supported on a distal end of the tubular body 64. The tubular body 64 includes one or more slots 64a (only one shown in FIG. 15) defined in a distal end portion of tubular body 64. The slots 64a may be defined on opposed sides of a distal end portion of tubular body 64 in mirrored relationship. The actuation assembly 66 includes a lever 66a pivotally coupled to the handle assembly 62. A linkage 66b having a first end coupled to the lever 66a and a second end coupled to a proximal end of a drive shaft 66c. The actuation assembly 66 includes a spring 66d mounted in the handle assembly 62 about the drive shaft 66c. The actuation assembly 66 further includes coupling fingers 66f, 66g that are cooperatively engaged with a distal end of the drive shaft 66c and pivotally coupled to the distal end portion of the tubular body 64.

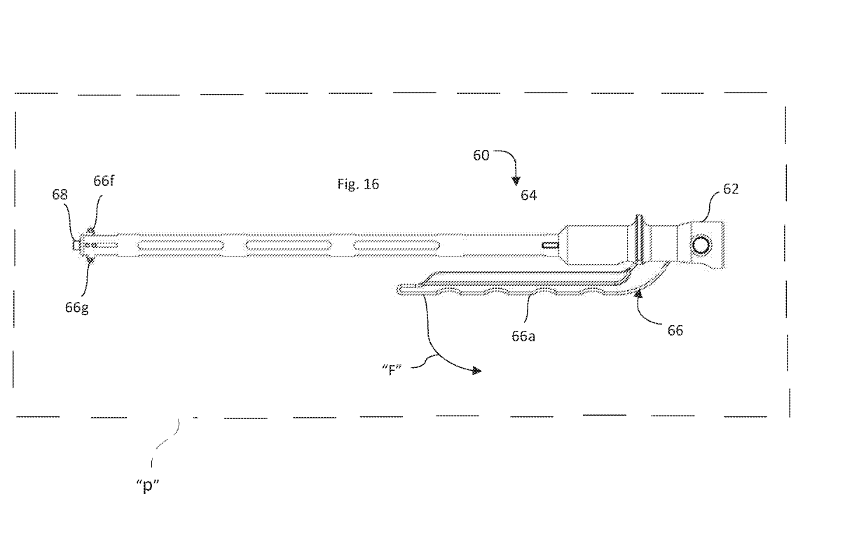

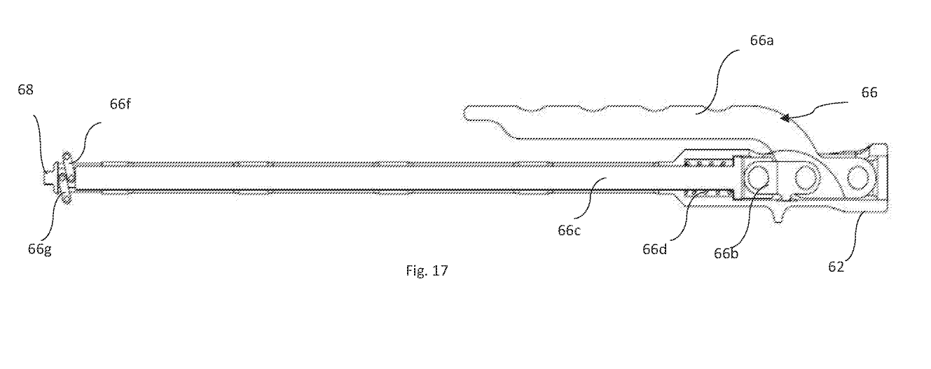

In use, the tower removal instrument 60 may be coupled to the proximal end 30a of the reduction tower 30 to selectively separate the reduction tower 30 from the bone anchor 20, for example, after a spinal rod “R” is reduced into the bone anchor 20 as described herein. In particular, the distal tip 68 of the tower removal instrument 60 may be received within the proximal end 30a of the reduction tower 30 and positioned so that the slots 64a of the tubular body 64 of the tower removal instrument 60 are aligned with the slots 33a of the reduction tower 30. Once the slots 64a of the tubular body 64 of the tower removal instrument 60 are aligned with the slots 33a of the reduction tower 30, the lever 66a of the tower removal instrument 60 is pivoted through a plane “P” extending longitudinally through the tower removal instrument 60. The pivoting movement of the lever 66a of the tower removal instrument 60, as indicated by arrow “E,” transitions the tower removal instrument 60 from the open position (FIG. 15) to the closed position (FIGS. 16 and 17). As the lever 66a of the tower removal instrument 60 pivots, the linkage 66b of the tower removal instrument 60 pivots towards the handle assembly 62 of the tower removal instrument 60 and translates the drive shaft 66c of the tower removal instrument 60 distally through the tubular body 64 of the tower removal instrument 60, thereby compressing the spring 66d of the tower removal instrument 60 and pivoting the coupling fingers 66f, 66g of the tower removal instrument 60 out of the slots 64a of the tubular body 64 of the tower removal instrument 60.

In the closed position of the tower removal instrument 60 (FIGS. 16 and 17), the coupling fingers 66f, 66g of the tower removal instrument 60 extend through the slots 64a of the tubular body 64 of the tower removal instrument 60 and into the slots 33a of the reduction tower 30 to couple the tower removal instrument 60 to the reduction tower 30. The tower removal instrument 60 can then be rotated about a longitudinal axis “X” defined by the tower removal instrument 60 to simultaneously rotate the reduction tower 30 about the bone anchor 20, thereby disengaging the reduction tower 30 from the bone anchor 20 so that the reduction tower 30 can be separated from the bone anchor 20 with the tower removal instrument 60. The lever 66a of the tower removal instrument 60 can then be released so that the spring 66d of the tower removal instrument 60 can urge the drive shaft 66d proximally whereby the coupling fingers 66f, 66g of the tower removal instrument 60 pivot back into the slots 64a of the tubular body 64 of the tower removal instrument 60 and whereby the linkage 66b and lever 66a of the tower removal instrument 60 pivot back to the open position, as indicated by arrow “F.”

Any of the presently disclosed embodiments, or components thereof, can be formed of any suitable material or combinations of materials such as mixed metallic materials like titanium alloy and cobalt-chromium.

Any of the presently disclosed embodiments, or components thereof can be formed using any suitable technique such as welding, fastening, machining, molding, etc. In some embodiments, one or more of the components can be secured together using any suitable technique such as welding, fastening, machining, molding, etc.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

1. A spinal rod reduction system, comprising:
a bone anchor including a housing and a threaded shank extending from the housing, the housing defining an opening configured to receive a spinal rod therein; and
a reduction tower having a pair of arms releasably attachable to the housing of the bone anchor, the pair of arms defining first and second elongated slots between the pair of arms, the first and second elongated slots configured to receive a spinal rod therethrough and direct a spinal rod toward the opening of the housing of the bone anchor, the first elongated slot positioned in opposed relation to the second elongated slot, the first elongated slot longer than the second elongated slot.

2. The spinal rod reduction system of claim 1, wherein the first and second elongated slots are aligned with the opening of the housing while the reduction tower is attached to the bone anchor.

3. The spinal rod reduction system of claim 1, wherein the bone anchor is configured to receive a set screw to selectively secure a spinal rod within the opening of the housing.

4. The spinal rod reduction system of claim 1, further including a rod reducer that selectively couples to the reduction tower to reduce a spinal rod along the first and second elongated slots and into the opening of the housing.

5. The spinal rod reduction system of claim 4, wherein the reduction tower includes a threaded proximal end configured to threadably engage the rod reducer.

6. The spinal rod reduction system of claim 1, further including a spinal rod.

7. The spinal rod reduction system of claim 1, further including a tower removal instrument engagable with the reduction tower and configured to separate the reduction tower from the bone anchor.

8. A spinal rod reduction apparatus, comprising:
an elongated body member;
a pair of arms extending distally from the elongated body member; and
first and second elongated slots defined between the pair of arms, the first and second elongated slots configured to receive a spinal rod therethrough, the first elongated slot positioned in opposed relation to the second elongated slot, the first elongated slot longer than the second elongated slot.

9. The spinal rod reduction apparatus of claim 8, wherein the pair of arms is configured to couple to a bone anchor defining an opening for receiving a spinal rod therein, the first and second elongated slots positioned to align with the opening of the bone anchor while the pair of arms is coupled to the bone anchor.

10. The spinal rod reduction apparatus of claim 8, wherein the pair of arms extends to a distal end that is engagable with an outer surface of the bone anchor.

11. The spinal rod reduction apparatus of claim 8, wherein the pair of arms are positioned to receive a set screw between the pair of arms, the set screw advanceable along an inner surface of the pair of arms to reduce a spinal rod received between the pair of arms.

12. The spinal rod reduction apparatus of claim 8, wherein a proximal end of the elongated body member is selectively engagable with a rod reducer.

13. The spinal rod reduction apparatus of claim 12, wherein the proximal end of the elongated body member is threaded.

14. The spinal rod reduction apparatus of claim 8, wherein the pair of arms and the body member are integrally formed.

15. A method of reducing a spinal rod, the method comprising:
securing a bone anchor and a reduction tower to a vertebra;
positioning the spinal rod through a first elongated slot of the reduction tower;
advancing the spinal rod along the first elongated slot toward a second elongated slot;
advancing the spinal rod through the second elongated slot; and
advancing the spinal rod along the first and second elongated slots and towards the bone anchor.

16. The method of claim 15, further including securing the reduction tower to the bone anchor before the bone anchor is secured to the vertebra.

17. The method of claim 15, further including securing a rod reducer to the reduction tower.

18. The method of claim 17, further including advancing the spinal rod along the reduction tower by rotating a proximal end of the rod reducer around a proximal end of the reduction tower.

19. The method of claim 15, further including separating the reduction tower from the bone anchor after the spinal rod is reduced into the bone anchor.

20. The method of claim 19, wherein separating the reduction tower from the bone anchor includes coupling a tower removal instrument to the reduction tower.
